(19) United States
(12) Patent Application Publication
(10) Pub. No.: US 2014/0142722 A1
(43) Pub. Date: May 22, 2014

(54) MODULARIZED KNEE JOINT CUSHIONING STRUCTURE

(71) Applicant: KEN DALL ENTERPRISE CO., LTD., New Taipei City (TW)

(72) Inventor: CHIH HSUAN LIANG, New Taipei City (TW)

(73) Assignee: KEN DALL ENTERPRISE CO., LTD., New Taipei City (TW)

(21) Appl. No.: 13/680,118

(22) Filed: Nov. 19, 2012

(51) Int. Cl. A61F 2/38

(52) U.S. Cl. CPC A61F 2/38
USPC 623/46

(57) ABSTRACT

A modularized knee joint cushioning structure includes a top joint seat connectable to a thigh, a friction adjustment assembly including an adjustment screw and coupled, in combination with a spring assembly, to the top joint seat and a bottom joint seat, a caliper forming an axle hole having a circumference forming a gap extending to the outside and including a bearing surface formed on an outside surface thereof, a main body having a main axle connecting the caliper and the top joint seat, and a bottom joint seat having a top end forming, at one side thereof, a brake block and coupled to the main body and the spring assembly. The spring assembly may alternatively used with a fixing assembly to limit the spring force of the spring assembly. The spring assembly is replaceable by a hydraulic assembly or a pneumatic assembly to form alternative cushioning structures.

### TECHNICAL FIELD OF THE INVENTION

The present invention generally relates to a knee joint cushioning structure of an artificial limb, and more particularly to a modularized knee joint cushioning structure.

### DESCRIPTION OF THE PRIOR ART

With the progress of medical practice and technology following the development of medical science, various cushioning means has been available for the knee joint of general artificial limb, such as spring, hydraulic, and pneumatic structures. The cushioning structures of different types provide different shock absorption and wearing comfortableness.

Each of these cushioning structures is composed of a plurality of components assembled together. When a primary cushioning component fails, a technician must take the whole structure apart to proceed with replacement and maintenance. This is time-consuming. The cushioning structure is generally fixed and cannot be changed as desired by a user. Consequently, a user, who wishes to replace with a different cushioning structure, must purchase a new knee joint that adopts the cushioning structure. This increases economic loading of the user.

In view of the shortcomings of the conventional knee joint of artificial limb that requires an extended time of maintenance, has excessive complexity, and imposes additional economic loading, the present invention aims to provide a cushioning structure of knee joint of artificial limb that overcomes the above shortcomings.

### SUMMARY OF THE INVENTION

The primary object of the present invention is to provide a knee joint comprising a modularized replaceable cushioning structure to provide an artificial limb user with diversified cushioning structures and reduce maintenance time and economic loading.

To achieve the above object, the present invention provides a modularized knee joint cushioning structure, which comprises a top joint seat, which is connectable to a thigh; a friction adjustment assembly, which comprises an adjustment screw mounted to a side thereof; a spring assembly, which is coupled, in combination with the friction adjustment assembly, to the top joint seat and a bottom joint seat by coupling mean; a caliper, which forms an axle hole having a circumference forming a gap extending to the outside and also comprises a bearing surface formed on an outside surface thereof; a main body, which comprises a main axle connecting the caliper and the top joint seat; and a bottom joint seat, which has a top end forming, at one side thereof, a brake block and is coupled by coupling means to the main body and the spring assembly. The modularized knee joint cushioning structure is constructed in such a way that the spring assembly can be replaced by a fixing assembly, a hydraulic assembly, or a pneumatic assembly to provide three additional structures and thus achieving the purposes of component substitution.

The present invention provides the following advantages:

(1) When an artificial limb user wishes to use a knee joint of a different cushioning structure, he can select among a spring assembly, a fixing assembly, a hydraulic assembly, and a pneumatic assembly as he needs and has a professional technician to make the replacement or upgrading for him. Further, the maintenance time for the module, the complexity of components, and cost are all greatly reduced. There is no need to entirely replace the whole knee joint and the cushioning structure is of improved flexibility.

(2) When the spring assembly is incorporated in the modularized knee joint cushioning structure, a friction adjustment assembly can be used additionally in order to limit and maintain the spring force of the spring assembly.

(3) When the modularized knee joint cushioning structure is set in operation in walking, the bearing surface of the caliper is caused to engage the brake block of the bottom joint seat to pinch the main axle to provide a safe braking operation in combination with cushioning effected by the elastic body.

The foregoing objectives and summary provide only a brief introduction to the present invention. To frilly appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of a structure according to the present invention.

FIG. 1A is an exploded view of the structure according to the present invention.

FIG. 1B is a perspective view of the structure according to the present invention taken at a different angle.

FIG. 1C is a side elevational view of the structure according to the present invention.

FIG. 2 is an exploded view of a structure according to another embodiment of the present invention.

FIG. 3 is an exploded view of a structure according to a further embodiment of the present invention.

FIG. 4 is an exploded view of a structure according to yet a further embodiment of the present invention.

FIG. 5 is a schematic view illustrating a standing and walking condition of the structure according to the present invention.

FIG. 5A is a cross-sectional view of the structure of the present invention in the standing and walking condition.

FIG. 5B is an enlarged cross-sectional view of a portion of the structure of the present invention in the standing and walking condition.

FIG. 5C is a cross-sectional view of the structure of the present invention in the standing and walking condition.

FIG. 5D is a cross-sectional view of the structure of the present invention in the standing and walking condition.

FIG. 6 shows a bending and braking condition of the structure according to the present invention.

FIG. 6A is a cross-sectional view showing the bending and braking condition of the structure according to the present invention.

FIG. 6B is another cross-sectional view showing the bending and braking condition of the structure according to the present invention.

FIG. 6C is an enlarged cross-sectional view of a portion of the structure according to the present invention in the bending and braking condition.

FIG. 6D is a cross-sectional view showing the bending and braking condition of the structure according to the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

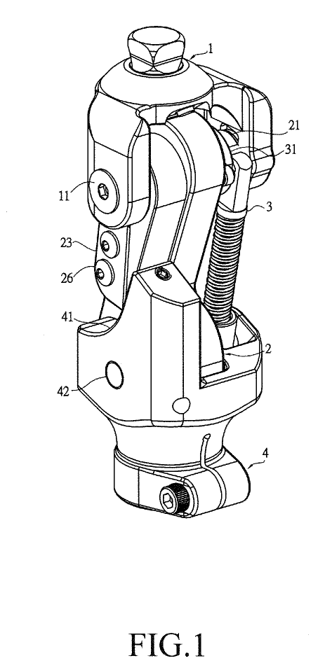

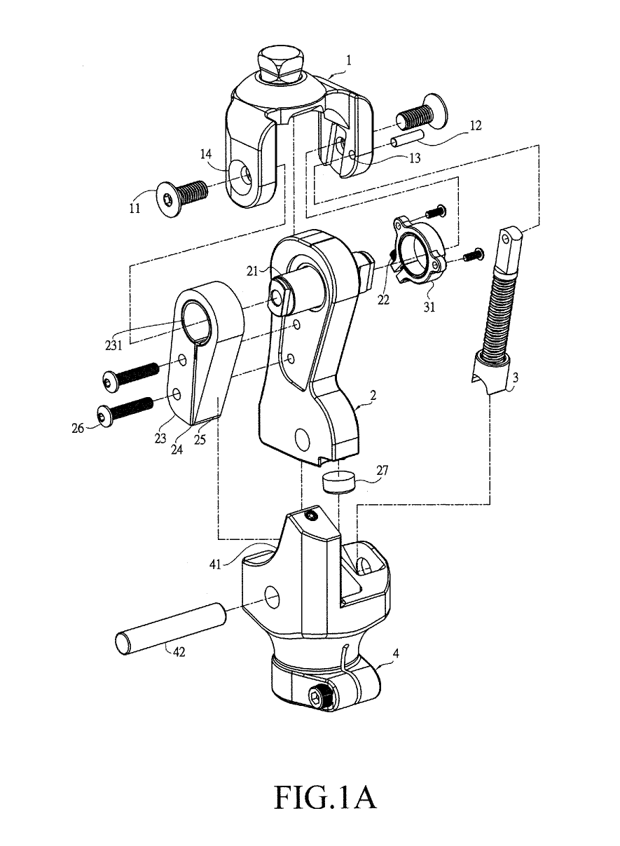

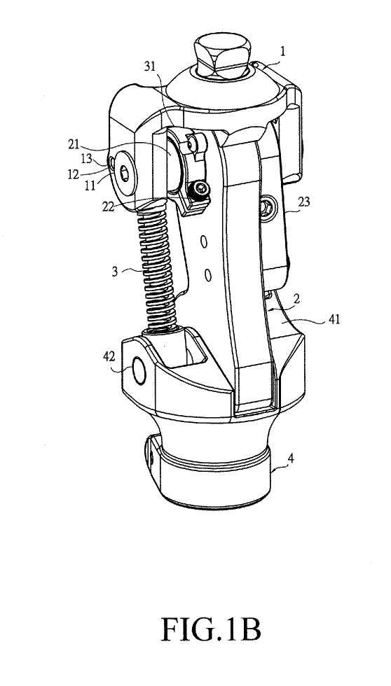

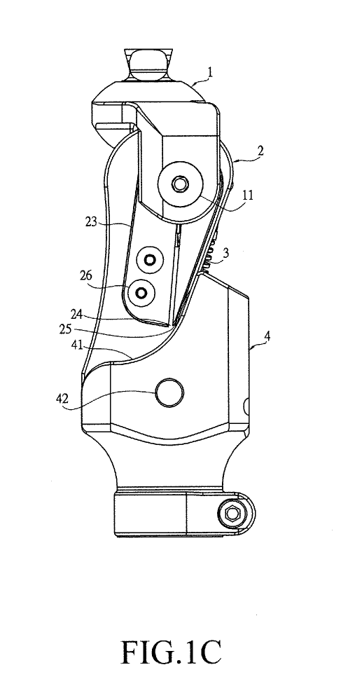

Structure and functionality of the present invention will be described by way of preferred embodies, with reference to the attached drawings, to provide a throughout understanding of the present invention. The present invention provides a modularized knee joint cushioning structure, which, as shown in FIGS. 1-5D, is generally composed of components including a top joint seat 1, a main body 2, a caliper 23, a spring assembly 3, a friction adjustment assembly 31, a fixing assembly 32, a hydraulic assembly 33, a pneumatic assembly 34, and a bottom joint seat 4.

As shown in FIGS. 1-1C, the top joint seat 1 has a top end connectable to a thigh, two side walls forming apertures 13 and threaded holes 14, and a lower end coupled to the main body 2 by coupling means. The coupling means comprises threaded connection effected with coupling bolts 11. The friction adjustment assembly 31 comprises an adjustment screw 22 mounted to a side thereof. With the friction adjustment assembly 31 being fit over the main axle 21, the adjustment screw 22 functions to adjust tightness of clamping of the friction adjustment assembly 31 so as to limit the spring force of the spring assembly 3. The spring assembly 3, in combination with the friction adjustment assembly 31, is coupled by coupling means to the top joint seat 1. The coupling means comprises a generally known solution of extending a shaft 12 therethrough. The caliper 23 forms an axle hole 231, which has a circumference forming a gap 24 extending to the outside and also comprises a bearing surface 25 formed on an outside surface thereof. The main body 2 comprises a main axle 21 connecting the caliper 23 and the top joint seat 1. The bottom joint seat 4 has a top end forming, at one side thereof, a brake block 41, with an elastic body 27 being received between the main body 2 and the bottom joint seat 4, and is coupled to the main body 2 and the spring assembly 3 by coupling means that comprises a shaft 42 extending therethrough.

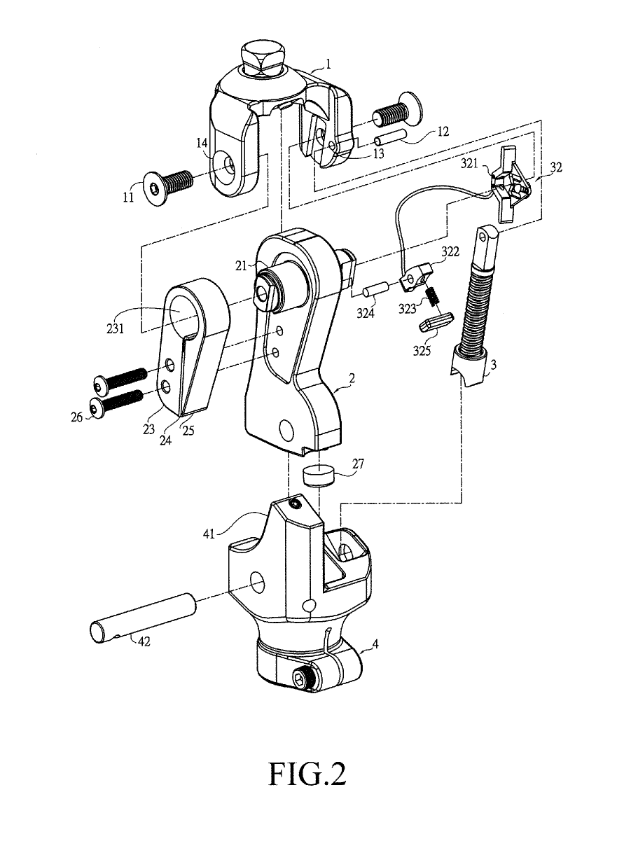

Another embodiment of the present invention is illustrated in FIG. 2, in which a fixing assembly 32 is used to replace the friction adjustment assembly 31 of the previous embodiment, and structurally comprises a top joint seat 1, which has a top end connectable to a thigh; a fixing assembly 32, which comprises a hand puller assembly 321 connected to a fixer 322 that comprises a spring 323 in combination with a spring holder 325 with a shaft 324 extending in the fixer 322; a spring assembly 3, which is coupled, in combination with the fixing assembly 32, by coupling means to the top joint seat 1 and the bottom joint seat 4; a caliper 23, which forms an axle hole 231 that has a circumference forming a gap 24 extending to the outside and also comprises a bearing surface 25 formed on an outside surface thereof; a main body 2, which comprises a main axle 21 connecting the caliper 23 and the top joint seat 1 and has one side connected to the fixing assembly 32; a bottom joint seat 4, which has a top end forming, at one side thereof, a brake block 41 and is coupled by coupling means to the main body 2 and the spring assembly 3.

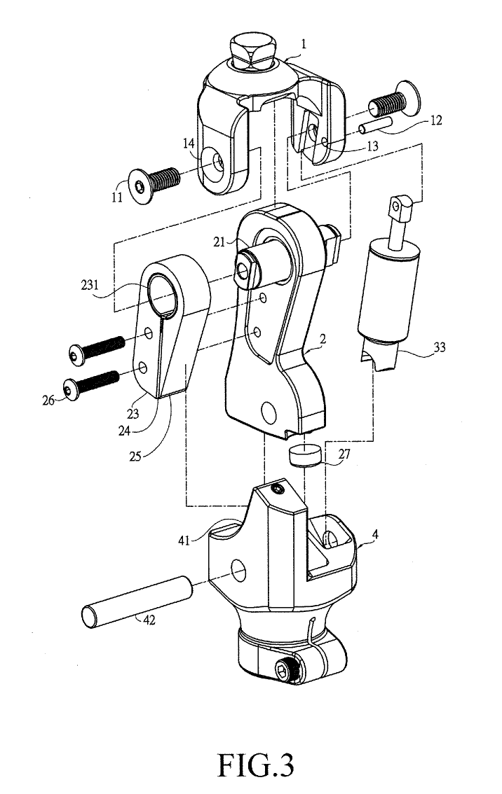

A further embodiment of the present invention is illustrated in FIG. 3, in which a hydraulic assembly 33 is used to replace the spring assembly 3 and the friction adjustment assembly 31 operating in combination therewith, and structurally comprises a top joint seat 1, which has a top end connectable to a thigh, two side walls forming apertures 13 and threaded holes 14, and a lower end coupled to the main body 2 by coupling means that comprises threaded connection effected with coupling bolts 11; a hydraulic assembly 33, which is coupled to the top joint seat 1 and a bottom joint seat 4 by coupling means that comprises a generally known solution of extending a shaft 12 therethrough; a caliper 23, which forms an axle hole 231 that has a circumference forming a gap 24 extending to the outside and also comprises a bearing surface 25 formed on an outside surface thereof; a main body 2, which comprises a main axle 21 connecting the caliper 23 and the top joint seat 1; a bottom joint seat 4, which has a top end forming, at one side thereof, a brake block 41, with an elastic body 27 being received between the main body 2 and the bottom joint seat 4 and is coupled to the main body 2 and the hydraulic assembly 33 by coupling means that comprises a shaft 42 extending therethrough.

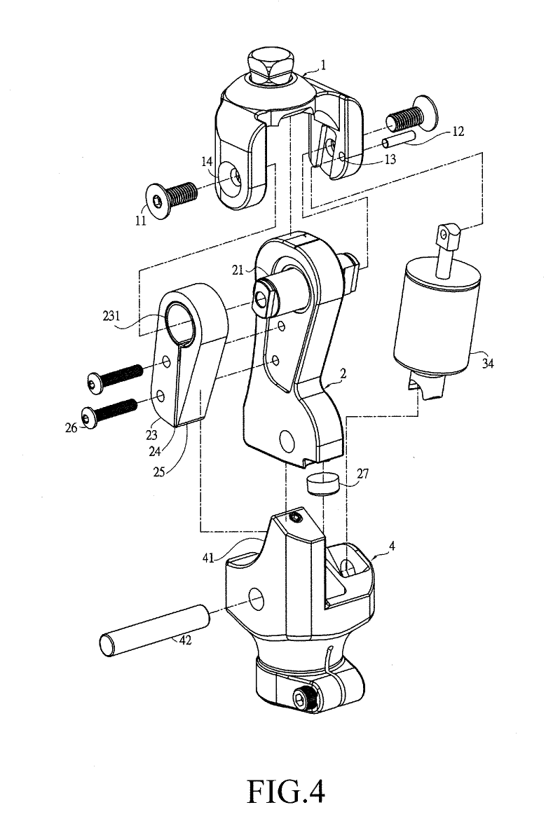

A further embodiment of the present invention is illustrated in FIG. 4, in which a pneumatic assembly 34 is used to replace the hydraulic assembly 33, and structurally comprises a top joint seat 1, which has a top end connectable to a thigh, two side walls forming apertures 13 and threaded holes 14, and a lower end coupled to the main body 2 by coupling means that comprises threaded connection effected with coupling bolts 11; a pneumatic assembly 34, which is coupled to the top joint seat 1 and a bottom joint seat 4 by coupling means that comprises a generally known solution of extending a shaft 12 therethrough; a caliper 23, which forms an axle hole 231 that has a circumference forming a gap 24 extending to the outside and also comprises a bearing surface 25 formed on an outside surface thereof; a main body 2, the main body 2, which comprises a main axle 21 connecting the caliper 23 and the top joint seat 1; a bottom joint seat 4, which has a top end forming, at one side thereof, a brake block 41, with an elastic body 27 received between the main body 2 and the bottom joint seat 4, and is coupled to the main body 2 and the pneumatic assembly 34 by coupling means that comprises a shaft 42 extending therethrough.

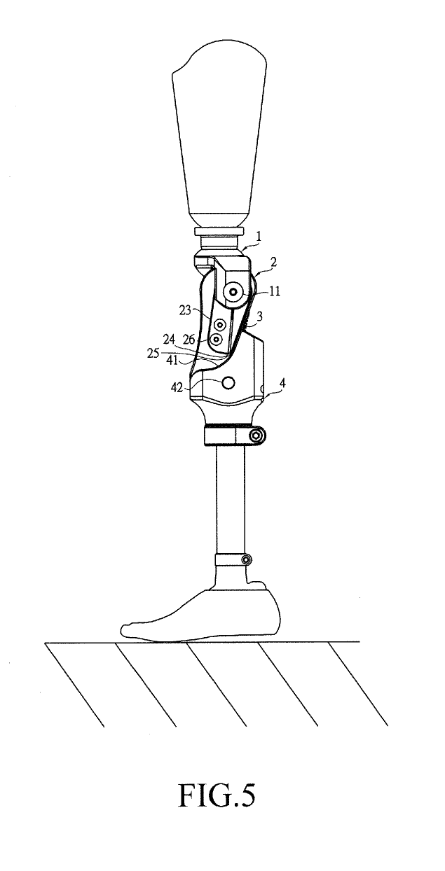

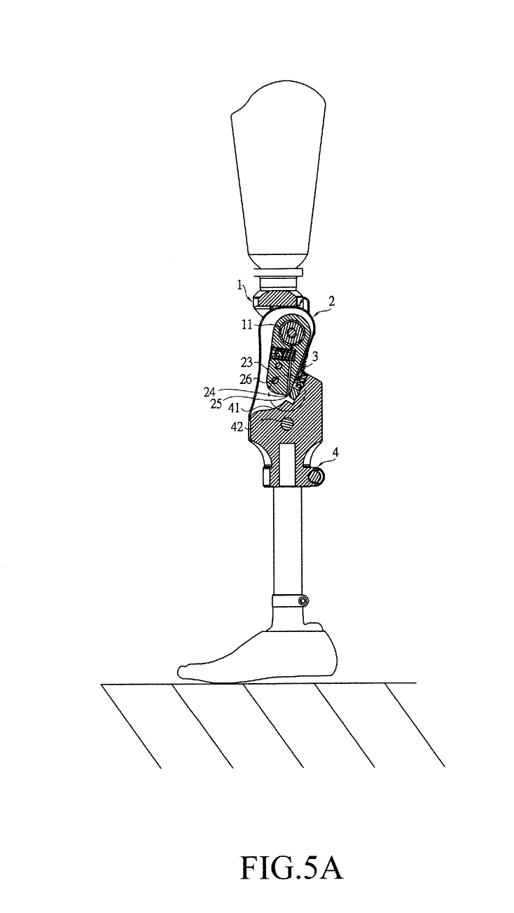

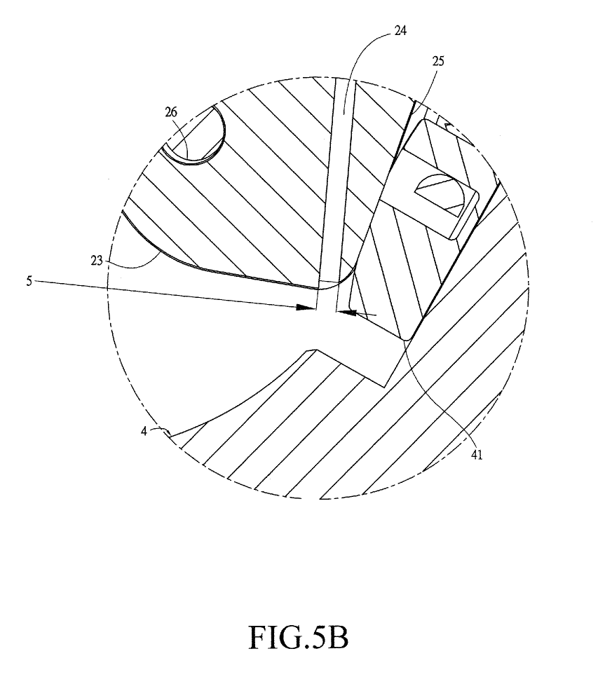

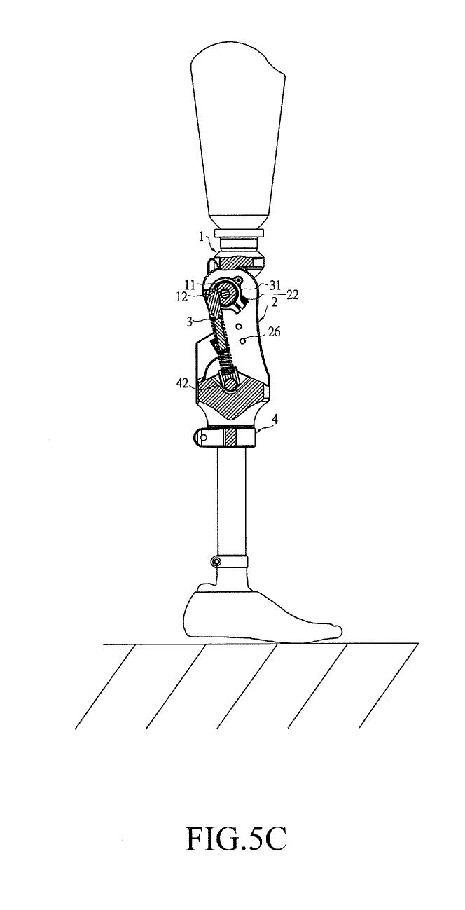

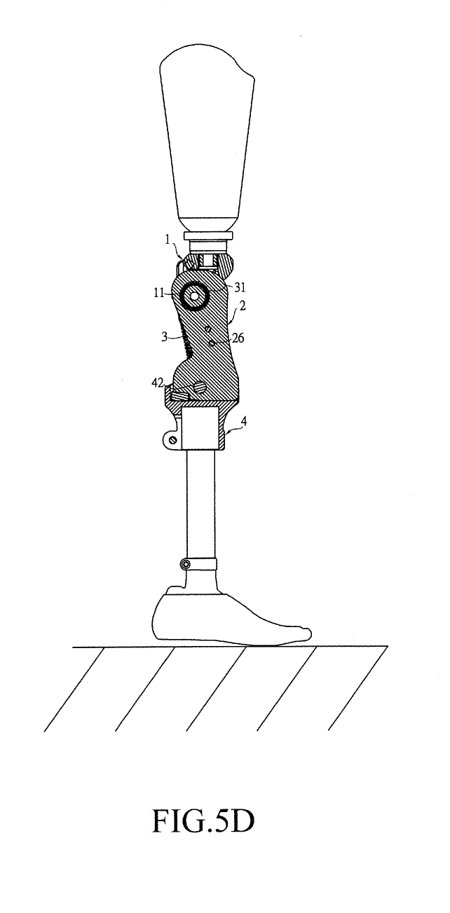

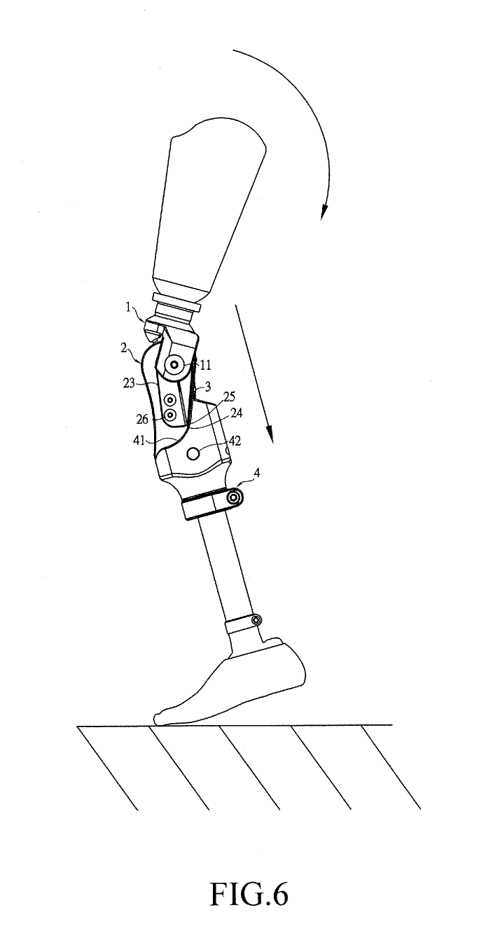

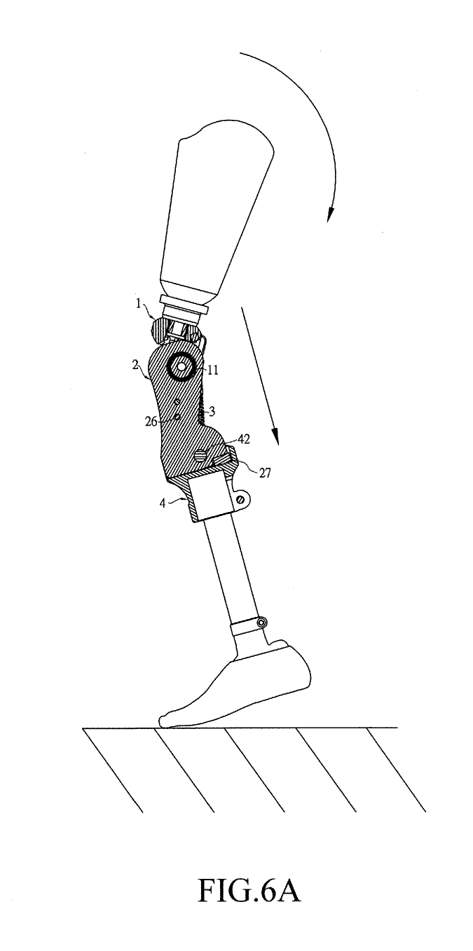

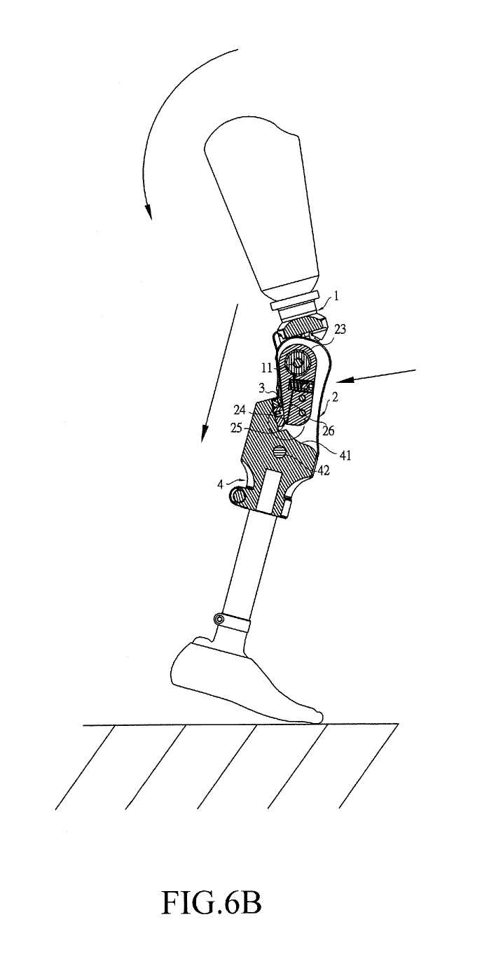

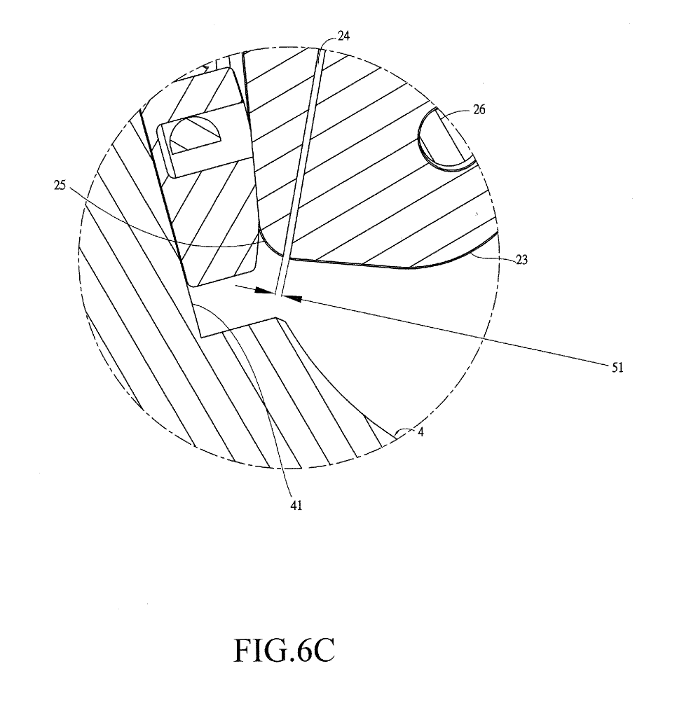

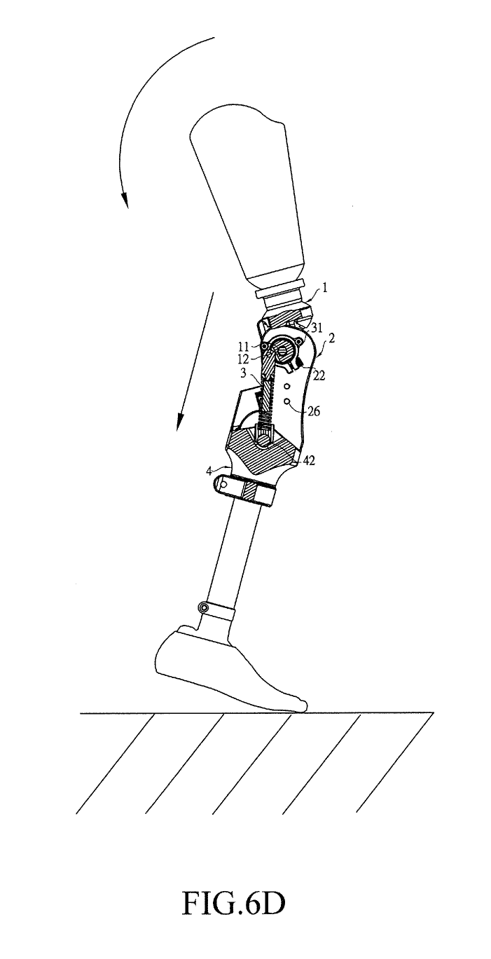

Referring to FIGS. 1-4, the present invention provides a modularized knee joint cushioning structure, which comprises a combined structure of the spring assembly 3 in combination with the friction adjustment assembly 31, or the fixing assembly 32, or the hydraulic assembly 33, the pneumatic assembly 34 that is arranged in such a way that the top thereof is coupled in the top joint seat 1 with the extension of the shaft 12 therethrough and the bottom thereof is coupled in the bottom joint seat 4 with the extension of the shaft 42 therethrough so that replacement of the cushioning structure of the knee joint can be done easily and the time and money spent in a maintenance operation are reduced due to modularization. Referring to FIGS. 5-5D, in a standing and walking condition, although the bearing surface 25 of the caliper is in contact with the brake block 41 of the bottom joint seat 4, yet the gap spacing A5 of the caliper 23 does not allow the gap 24 to generate a pinching effect on the main axle 21. The spring assembly 3 on the opposite side is coupled and fixed to the top joint seat 1 and the bottom joint seat 4 and the spring assembly 3 is not compressed. As shown in FIGS. 6-6D, in a bending and braking condition, the bearing surface 25 of the caliper 23 and the brake block 41 of the bottom joint seat 4 is affected by the bending to have the gap spacing B51 of the caliper 23 causing the gap 24 to clamp the main axle 21 and thus achieving a braking effect. The spring assembly 3 on the opposite side is compressed, but due to the main axle 21 being clamped by the gap 24 of the caliper 23, so that the knee joint is entirely kept in a braked condition. When the structure resumes the standing and walking condition, the spring assembly 3 restores the uncompressed condition to provide the cushioning effect. When the standing and walking condition and the bending and braking condition are applied alternately, the braking and cushioning functions also cyclically effected accordingly. It is only when the spring assembly 3 is used, the friction adjustment assembly 31 is needed and the friction adjustment assembly 31 can be replaced by the fixing assembly 32 that works with the spring assembly 3. When a hydraulic assembly 33 or a pneumatic assembly 34 is used alternatively, due to they provide a restoration function by themselves, there is no need to incorporate the friction adjustment assembly 31 for adjustment and they also provides a function of modularization of cushioning structure for replacement.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A modularized knee joint cushioning structure, comprising:
a top joint seat, which has a top end connectable to a thigh;
a friction adjustment assembly, which comprises an adjustment screw mounted to a side thereof;
a spring assembly, which is coupled, in combination with the friction adjustment assembly, to the top joint seat and a bottom joint seat by coupling mean;
a caliper, which forms an axle hole having a circumference forming a gap extending to the outside and also comprises a bearing surface formed on an outside surface thereof;
a main body, which comprises a main axle connecting the caliper and the top joint seat; and
a bottom joint seat, which has a top end forming, at one side thereof, a brake block and is coupled by coupling means to the main body and the spring assembly.

2. The modularized knee joint cushioning structure as claimed in claim 1, wherein the friction adjustment assembly is used in combination with the spring assembly to limit spring force of the spring assembly.

3. The modularized knee joint cushioning structure as claimed in claim 1, wherein the bearing surface of the caliper is engageable with the brake block of the bottom joint seat in a walking condition to pinch the main axle and to induce a braking operation in combination with cushioning effected by the elastic body.

4. The modularized knee joint cushioning structure as claimed in claim 1, wherein the spring assembly is selected by a user and is replaceable by a professional technician to achieve the purposes of modularization.

5. A modularized knee joint cushioning structure, comprising:
a top joint seat, which has a top end connectable to a thigh;
a fixing assembly, which comprises a hand puller assembly connected to a fixer that comprises a spring in combination with a spring holder with a shaft extending in the fixer;
a spring assembly, which is coupled, in combination with the fixing assembly, by coupling means to the top joint seat and a bottom joint seat;
a caliper, which forms an axle hole that has a circumference forming a gap extending to the outside and also comprises a bearing surface formed on an outside surface thereof;
a main body, which comprises a main axle connecting the caliper and the top joint seat and has one side connected to the fixing assembly; and
a bottom joint seat, which has a top end forming, at one side thereof, a brake block and is coupled by coupling means to the main body and the spring assembly.

6. The modularized knee joint cushioning structure as claimed in claim 5, wherein the bearing surface of the caliper is engageable with the brake block of the bottom joint seat in a walking condition to pinch the main axle and to induce a braking operation in combination with cushioning effected by the elastic body.

7. The modularized knee joint cushioning structure as claimed in claim 5, wherein the fixing assembly is selected by a user and is replaceable by a professional technician to achieve the purposes of modularization.

8. A modularized knee joint cushioning structure, comprising:
a top joint seat, which has a top end connectable to a thigh;
a hydraulic assembly, which is coupled to the top joint seat and a bottom joint seat by coupling means;
a caliper, which forms an axle hole that has a circumference forming a gap extending to the outside and also comprises a bearing surface formed on an outside surface thereof;
a main body, which comprises a main axle connecting the caliper and the top joint seat; and
a bottom joint seat, which has a top end forming, at one side thereof, a brake block and is coupled to the main body and the hydraulic assembly by coupling means.

9. The modularized knee joint cushioning structure as claimed in claim 8, wherein the bearing surface of the caliper is engageable with the brake block of the bottom joint seat in a walking condition to pinch the main axle and to induce a braking operation in combination with cushioning effected by the elastic body.

10. The modularized knee joint cushioning structure as claimed in claim 8, wherein the hydraulic assembly is selected by a user and is replaceable by a professional technician to achieve the purposes of modularization.

11. A modularized knee joint cushioning structure, comprising:
a top joint seat, which has a top end connectable to a thigh;
a pneumatic assembly, which is coupled to the top joint seat and a bottom joint seat by coupling means;
a caliper, which forms an axle hole that has a circumference forming a gap extending to the outside and also comprises a bearing surface formed on an outside surface thereof;
a main body, which comprises a main axle connecting the caliper and the top joint seat; and
a bottom joint seat, which has a top end forming, at one side thereof, a brake block and is coupled to the main body and the pneumatic assembly by coupling means.

12. The modularized knee joint cushioning structure as claimed in claim 11, wherein the bearing surface of the caliper is engageable with the brake block of the bottom joint seat in a walking condition to pinch the main axle and to induce a braking operation in combination with cushioning effected by the elastic body.

13. The modularized knee joint cushioning structure as claimed in claim 11, wherein the pneumatic assembly is selected by a user and is replaceable by a professional technician to achieve the purposes of modularization.
